(19) United States
(12) Patent Application Publication
(10) Pub. No.: US 2025/0009959 A1
(43) Pub. Date: Jan. 9, 2025

(54) SOFT-START MECHANISM FOR WOUND MONITORING AND TREATMENT DEVICES

(71) Applicant: T.J.Smith and Nephew,Limited, Hull (GB)

(72) Inventors: Peter Georg Laitenberger, Cambridge (GB); Daryl Simon Peter Larkin, Beverley (GB); Felix Clarence Quintanar, Hull (GB); Roberto Damiao da Costa Rodrigues, Hull (GB)

(21) Appl. No.: 18/712,659

(22) PCT Filed: Nov. 22, 2022
(86) PCT No.: PCT/EP2022/082841

§ 371 Date: May 22, 2024

(30) Foreign Application Priority Data
Nov. 23, 2021 (GB) 2116857.0

(51) Int. Cl. A61M 1/00

(52) U.S. Cl. CPC A61M 1/962; A61M 2205/8206

(57) ABSTRACT

Electronic patient monitoring and treatment devices can include various loads (such as, pumps or sensors) that may need to be actively managed at startup to prevent a brownout condition and reset from occurring. To prevent the brownout and reset, an initial inrush current to the load can be limited by a soft-start circuit. In some cases, alternatively activating and deactivating a switch can packetize the energy transfer from a power source to the load and reduce the inrush current. Reducing the current can also reduce the required recovery time of the power source.

### TECHNICAL FIELD

Embodiments of the present disclosure relate to apparatuses, systems, and methods for managing power delivery in user treatment and monitoring devices.

### DESCRIPTION OF THE RELATED ART

The treatment of open or chronic wounds that are too large to spontaneously close or otherwise fail to heal by means of applying negative pressure to the site of the wound is well known in the art. Negative pressure wound therapy (“NPWT”) systems currently known in the art commonly involve placing a cover that is impermeable or semi-permeable to fluids over the wound, using various means to seal the cover to the tissue of the patient surrounding the wound, and connecting a source of negative pressure (such as a vacuum pump) to the cover in a manner so that negative pressure is created and maintained under the cover. It is believed that such negative pressures promote wound healing by facilitating the formation of granulation tissue at the wound site and assisting the body's normal inflammatory process while simultaneously removing excess fluid, which may contain adverse cytokines and/or bacteria. However, further improvements in NPWT are needed to fully realize the benefits of treatment.

### SUMMARY

Disclosed herein are methods of operating a negative pressure wound therapy system of any of the preceding paragraphs and/or any of the devices, apparatuses, or systems disclosed herein.

A negative pressure wound therapy system can include a battery. The system can include a power converter electrically connected to the battery and configured to increase voltage provided by the battery. The system can include a load configured to receive power from the power converter. The load can include a negative pressure source configured to provide negative pressure to a wound of a patient covered by a wound dressing. The system can include a switch electrically connected to the battery and the power converter and positioned between the battery and the power converter. The system can include control circuitry electrically connected to the switch and configured to alternate activating and deactivating the switch to reduce peak current provided by the battery. This can prevent a brownout condition and reset of the control circuitry.

The system of any of the preceding paragraphs and/or any of the systems or devices disclosed herein can include one or more of the following features. The system can include at least one energy storage component configured to be charged by the battery. The control circuitry can be configured to maintain the switch in an active state responsive to determining that the at least one energy storage component has been charged or responsive to an expiration of a threshold duration of time. The control circuitry can be configured to generate a pulse signal to alternate activating and deactivating the switch. A first duration of time during which the pulse signal is active can be determined from a threshold voltage drop across the battery. A second duration of time during which the pulse signal is inactive can be determined from a recovery time of the battery. The control circuitry can be configured to vary at least one of a frequency or duty cycle of the pulse signal. The power converter can be a DC-to-DC converter. The battery can be a coin cell battery. The switch can be a transistor. The control circuitry can be configured to pulse the switch at a predetermined frequency and duty cycle. The predetermined frequency can be associated with a resonant frequency of an LC circuit electrically connected to the power converter.

A patient monitoring and/or treatment device can include a power source. The system can include a power converter electrically connected to the power source and configured to increase voltage provided by the power source. The system can include a load configured to receive power from the power converter. The system can include a switch electrically connected to the power source and the power converter and positioned between the power source and the power converter. The system can include a control circuitry electrically connected to the switch and configured to alternate activating and deactivating the switch to reduce peak current provided by the power source.

The system of any of the preceding paragraphs and/or any of the systems or devices disclosed herein can include one or more of the following features. The power source can be a coin cell battery. The control circuitry can be configured to generate a pulse signal to alternate activating and deactivating the switch. A first duration of time during which the pulse signal is active can be determined from a threshold voltage drop across the power source. A second duration of time during which the pulse signal is inactive can be determined from a recovery time for the power source. The control circuitry can be configured to vary at least one of a frequency or duty cycle of the pulse signal. The power converter can be a DC-to-DC converter. The switch can be a transistor. The control circuitry can be configured to pulse the switch at a predetermined frequency and duty cycle. The predetermined frequency can be associated with a resonant frequency of an LC circuit electrically connected to the power converter.

A method of operating a patient monitoring and/or treatment device can include activating a switch to electrically connect a power source to a load. The method can include monitoring a voltage differential across the power source. The method can include, in response to determining that the voltage differential satisfies a maximum voltage drop threshold, deactivating the switch to disconnect the power source from the load. The method can include, activating the switch in response to determining that a recovery time satisfies a recovery time threshold. The method can be performed under control of a control circuitry of the device.

The method of any of the preceding paragraphs and/or any of the methods disclosed herein can include one or more of the following features. The method can include determining the recovery time from a pattern of current spikes associated with an instantaneous current draw from the power source. Activating and deactivating the switch can be performed using a pulse signal generated by the control circuitry. A first duration of time during which the pulse signal is active can be determined from a threshold voltage drop across the power source, and a second duration of time during which the pulse signal is inactive can be determined from a recovery time of the power source. The method can include varying at least one of a frequency or duty cycle of the pulse signal.

Any of the features, components, or details of any of the arrangements or embodiments disclosed in this application, including without limitation any of the apparatus embodiments and any of the negative pressure wound therapy embodiments disclosed herein, are interchangeably combinable with any other features, components, or details of any of the arrangements or embodiments disclosed herein to form new arrangements and embodiments.

### BRIEF DESCRIPTION OF THE DRAWINGS

FIGS. 1A-1C illustrate a wound dressing incorporating a source of negative pressure and/or other electronic components within the wound dressing;

FIGS. 2A-2B illustrate an electronics unit that may be incorporated into a wound dressing;

FIG. 3 is an exploded perspective view of an electronics assembly enclosing an electronics unit within a housing;

FIG. 4A illustrates a bottom perspective view of the electronics assembly of FIG. 3;

FIG. 4B illustrates a top perspective view of the electronics assembly of FIG. 3;

FIG. 5A is an exploded view of a wound dressing incorporating an electronics assembly within the wound dressing layers;

FIG. 5B illustrates a cross sectional layout of the material layers of a wound dressing incorporating an electronics assembly within the dressing;

FIGS. 6A-6B and 7A-7B illustrate components of an electronics assembly;

FIG. 8 illustrates a pump outlet mechanism;

FIG. 9 is a schematic representation of a circuit with a soft-start mechanism;

FIG. 10 illustrates components of a negative pressure wound therapy system;

FIG. 11 illustrates a user activity monitoring system that includes an activity monitoring device;

FIG. 12 illustrates a schematic of an activity monitoring device; and

FIG. 13 illustrates an activity monitoring device.

### DETAILED DESCRIPTION

Embodiments disclosed herein relate to apparatuses and methods of treating a wound with reduced pressure, including a source of negative pressure and wound dressing components and apparatuses. These apparatuses and components, including but not limited to wound overlays, backing layers, cover layers, drapes, sealing layers, spacer layers, absorbent layers, transmission layers, wound contact layers, packing materials, fillers and/or fluidic connectors are sometimes collectively referred to herein as dressings.

It will be appreciated that throughout this specification reference is made to a wound. It is to be understood that the term wound is to be broadly construed and encompasses open and closed wounds in which skin may be torn, cut or punctured or where trauma causes a contusion, or any other superficial or other conditions or imperfections on the skin of a patient or otherwise that benefit from reduced pressure treatment. A wound is thus broadly defined as any damaged region of tissue where fluid may or may not be produced. Examples of such wounds include, but are not limited to, abdominal wounds or other large or incisional wounds, either as a result of surgery, trauma, sterniotomies, fasciotomies, or other conditions, dehisced wounds, acute wounds, chronic wounds, subacute and dehisced wounds, traumatic wounds, flaps and skin grafts, lacerations, abrasions, contusions, burns, diabetic ulcers, pressure ulcers, stoma, surgical wounds, trauma and venous ulcers or the like.

It will be understood that embodiments of the present disclosure are generally applicable to use in NPWT or topical negative pressure (“TNP”) therapy systems. Briefly, negative pressure wound therapy assists in the closure and healing of many forms of “hard to heal” wounds by reducing tissue oedema; encouraging blood flow and granular tissue formation; removing excess exudate and may reduce bacterial load (and thus infection risk). In addition, the therapy allows for less disturbance of a wound leading to more rapid healing. TNP therapy systems may also assist on the healing of surgically closed wounds by removing fluid and by helping to stabilize the tissue in the apposed position of closure. A further beneficial use of TNP therapy can be found in grafts and flaps where removal of excess fluid is important and close proximity of the graft to tissue is required in order to ensure tissue viability.

As is used herein, reduced or negative pressure levels, such as −X mmHg, represent pressure levels relative to normal ambient atmospheric pressure, which can correspond to 760 mmHg (or 1 atm, 29.93 inHg, 101.325 kPa, 14.696 psi, 1013.25 mbar, etc.). Accordingly, a negative pressure value of −X mmHg reflects absolute pressure that is X mmHg below 760 mmHg or, in other words, an absolute pressure of (760-X) mmHg. In addition, negative pressure that is “less” or “smaller” than X mmHg corresponds to pressure that is closer to atmospheric pressure (such as, −40 mmHg is less than −60 mmHg). Negative pressure that is “more” or “greater” than −X mmHg corresponds to pressure that is further from atmospheric pressure (such as, −80 mmHg is more than −60 mmHg). In some cases, local ambient atmospheric pressure is used as a reference point, and such local atmospheric pressure may not necessarily be, for example, 760 mmHg.

The negative pressure range can be approximately −80 mmHg, or between about −20 mmHg and −200 mmHg. Note that these pressures are relative to normal ambient atmospheric pressure, which can be 760 mmHg. Thus, −200 mmHg would be about 560 mmHg in practical terms. In some cases, the pressure range can be between about −40 mmHg and −150 mmHg. Alternatively, a pressure range of up to −75 mmHg, up to −80 mmHg or over −80 mmHg can be used. Also in some cases a pressure range of below −75 mmHg can be used. Alternatively, a pressure range of over approximately −100 mmHg, or even −150 mmHg, can be supplied by the negative pressure apparatus.

### Wound Dressing

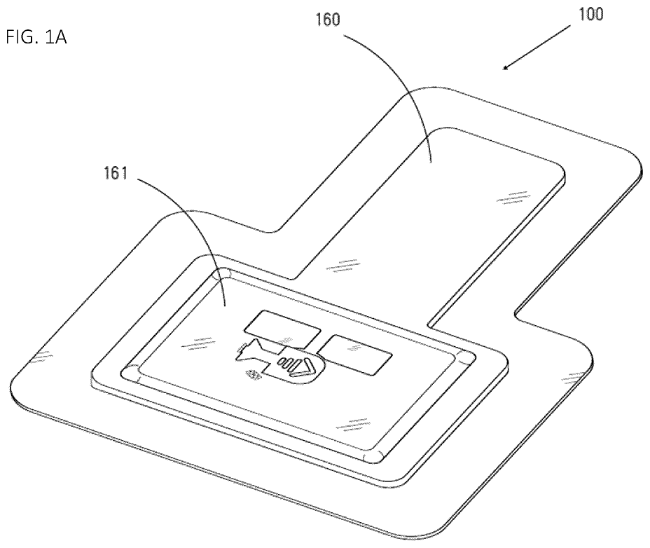

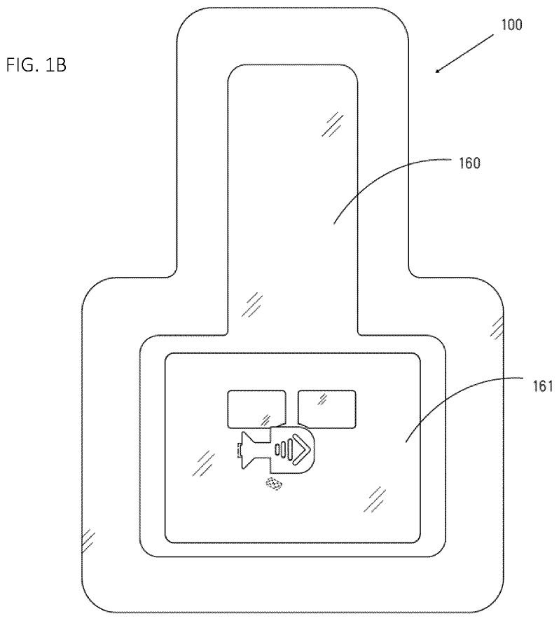

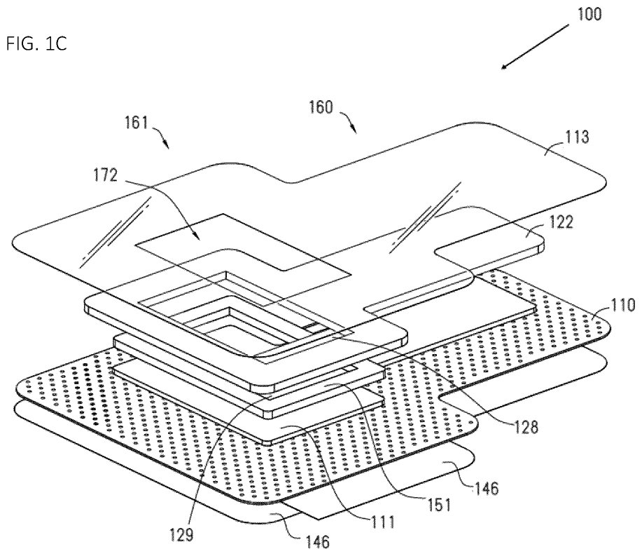

A source of negative pressure (such as a pump) and some or all other components of the TNP system, such as power source(s), sensor(s), connector(s), user interface component(s) (such as button(s), switch(es), speaker(s), screen(s), etc.) and the like, can be integral with the wound dressing. The material layers can include a wound contact layer, one or more absorbent layers, one or more transmission or spacer layers, and a backing layer or cover layer covering the one or more absorbent and transmission or spacer layers. The wound dressing can be placed over a wound and sealed to the wound with the pump and/or other electronic components contained under the cover layer within the wound dressing. The dressing can be provided as a single article with all wound dressing elements (including the pump) pre-attached and integrated into a single unit. A periphery of the wound contact layer can be attached to the periphery of the cover layer enclosing all wound dressing elements as illustrated in FIG. 1A-1C.

The pump and/or other electronic components can be configured to be positioned adjacent to or next to the absorbent and/or transmission layers so that the pump and/or other electronic components are still part of a single article to be applied to a patient. The pump and/or other electronics can be positioned away from the wound site. Although certain features disclosed herein may be described as relating to systems and method for controlling operation of a negative pressure wound therapy system in which the pump and/or other electronic components are positioned in or on the wound dressing, the systems and methods disclosed herein are applicable to any negative pressure wound therapy system or any medical device. FIGS. 1A-1C illustrate a wound dressing incorporating the source of negative pressure and/or other electronic components within the wound dressing. FIGS. 1A-1C illustrate a wound dressing 100 with the pump and/or other electronics positioned away from the wound site. The wound dressing can include an electronics area 161 and an absorbent area 160. The dressing can comprise a wound contact layer 110 (not shown in FIGS. 1A-1B) and a moisture vapor permeable film, cover layer or backing layer 113 positioned above the contact layer and other layers of the dressing. The wound dressing layers and components of the electronics area as well as the absorbent area can be covered by one continuous cover layer 113 as shown in FIGS. 1A-1C.

A layer 111 of porous material can be located above the wound contact layer 110. As used herein, the terms porous material, spacer, and/or transmission layer can be used interchangeably to refer to the layer of material in the dressing configured to distribute negative pressure throughout the wound area. This porous layer, or transmission layer, 111 allows transmission of fluid including liquid and gas away from a wound site into upper layers of the wound dressing. In particular, the transmission layer 111 preferably ensures that an open air channel can be maintained to communicate negative pressure over the wound area even when the absorbent layer has absorbed substantial amounts of exudates. The layer 111 should preferably remain open under the typical pressures that will be applied during negative pressure wound therapy as described above, so that the whole wound site sees an equalized negative pressure. The layer 111 may be formed of a material having a three dimensional structure. For example, a knitted or woven spacer fabric (for example Baltex 7970 weft knitted polyester) or a non-woven fabric could be used.

Further, one or more absorbent layers (such as layers 122, 151) for absorbing and retaining exudate aspirated from the wound can be utilized. A superabsorbent material can be used in the absorbent layers 122, 151. The one or more layers 122, 151 of absorbent material may be provided above the transmission layer 111. Since in use each of the absorbent layers experiences negative pressures, the material of the absorbent layer can be chosen to absorb liquid under such circumstances. The absorbent layers 122. 151 may comprise a composite comprising superabsorbent powder, fibrous material such as cellulose, and bonding fibers. The composite can be an airlaid, thermally-bonded composite.

The electronics area 161 can include a source of negative pressure (such as a pump) and some or all other components of the TNP system, such as power source(s), sensor(s), connector(s), user interface component(s) (such as button(s), switch(es), speaker(s), screen(s), etc.) and the like, that can be integral with the wound dressing. For example, the electronics area 161 can include a button or switch (shown in FIGS. 1A-1B as being covered by a pull tab). The button or switch can be used for operating the pump (such as, turning the pump on/off).

The electronics area 161 of the dressing can comprise one or more layers of transmission or spacer material and/or absorbent material and electronic components can be embedded within the one or more layers of transmission or spacer material and/or absorbent material. The layers of transmission or absorbent material can have recesses or cut outs to embed the electronic components within whilst providing structure to prevent collapse. As shown in FIG. 1C, recesses 128 and 129 can be provided in absorbent layers 151 and 122, respectively.

As used herein the upper layer, top layer, or layer above refers to a layer furthest from the surface of the skin or wound while the dressing is in use and positioned over the wound. Accordingly, the lower surface, lower layer, bottom layer, or layer below refers to the layer that is closest to the surface of the skin or wound while the dressing is in use and positioned over the wound. Additionally, the layers can have a proximal wound-facing face referring to a side or face of the layer closest to the skin or wound and a distal face referring to a side or face of the layer furthest from the skin or wound.

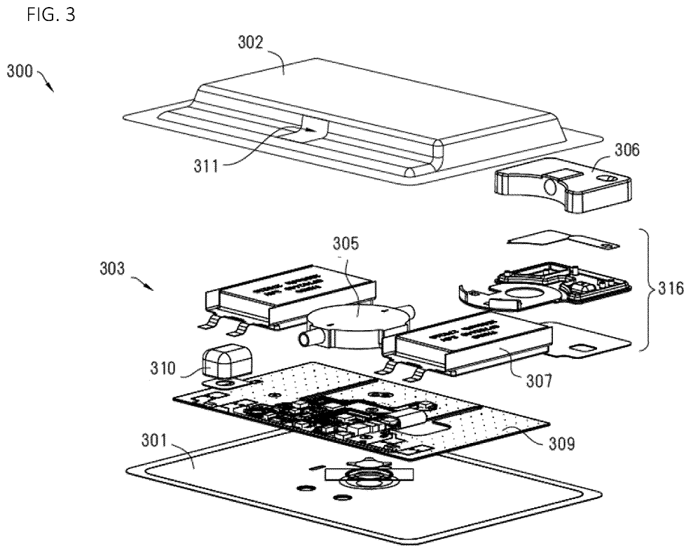

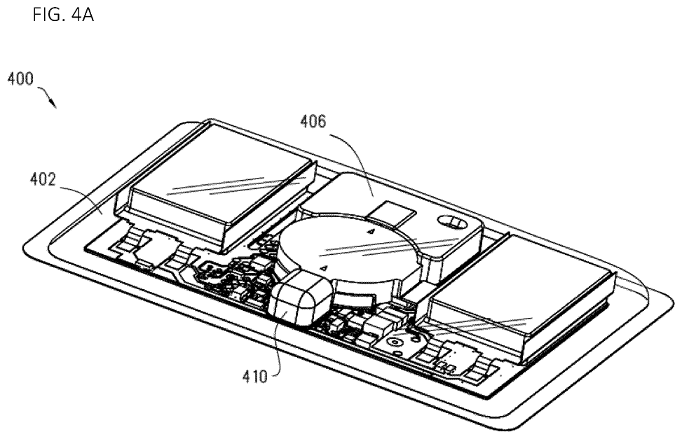

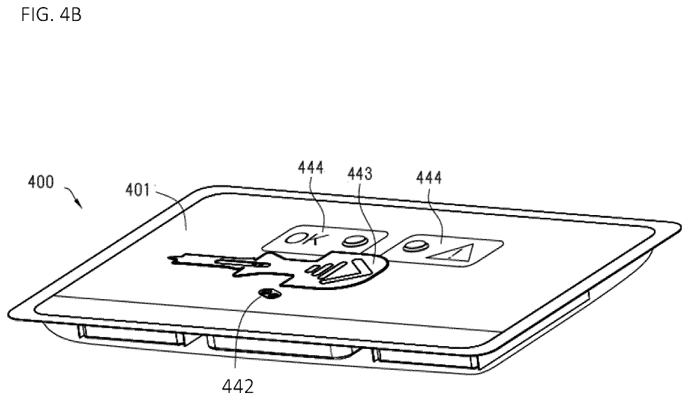

The cover layer may include a cutout 172 positioned over at least a portion of the aperture 128 in the absorbent layer 122 to allow access and fluid communication to at least a portion of the absorbent layers 122 and 151, transmission layer 111, and would contact layer 110 positioned below. An electronics assembly such as described below can be positioned in the apertures 128, 129, and 172 of the first and second absorbent material 151 and 122 and the cover layer 113. The electronics assembly can include a pump, power source, and a printed circuit board as described with reference to FIGS. 3 and 4A-4B.

Before use, the dressing can include one or more delivery layers 146 adhered to the bottom surface of the wound contact layer. The delivery layer 146 can cover adhesive or apertures on the bottom surface of the wound contact layer 110. The delivery layer 146 can provided support for the dressing and can assist in sterile and appropriate placement of the dressing over the wound and skin of the patient. The delivery layer 146 can include handles that can be used by the user to separate the delivery layer 146 from the wound contact layer 110 before applying the dressing to a wound and skin of a patient.

### Electronics Assembly Incorporated Within the Wound Dressing

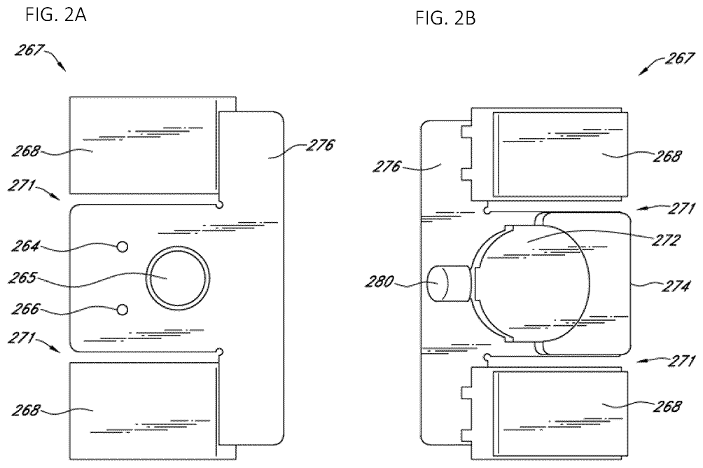

FIGS. 2A-2B illustrate an electronics unit 267 that can be incorporated into a wound dressing. FIG. 2A illustrates the top view of the electronics unit. FIG. 2B illustrates a bottom or wound facing surface of the electronics unit. The electronics unit 267 can include a pump 272 and one or more power sources 268, such as batteries. The electronics unit 267 can include a circuit board 276 configured to be in electrical communication with the pump 272 and/or power source 268. The circuit board 276 can be flexible or substantially flexible.

As illustrated in FIG. 2A, the electronics unit 267 can include single button or switch 265 on the upper surface of the unit. The single button or switch 265 can be used as an on/off button or switch to stop and start operation of the pump and/or electronic components. The electronics unit 267 can also include one or more vents or exhaust apertures 264 on the circuit board 276 for expelling the air exhausted from the pump. As shown in FIG. 2B, a pump outlet exhaust mechanism 274 (sometimes referred to as pump exhaust mechanism or pump outlet mechanism) can be attached to the outlet of the pump 272.

The electronics unit 267 can include a pump inlet protection mechanism 280 as shown in FIG. 2B positioned on the portion of the electronics unit closest to the absorbent area and aligned with the inlet of the pump 272. The pump inlet protection mechanism 280 is positioned between the pump inlet and the absorbent area or absorbent layer of the dressing. The pump inlet protection mechanism 280 can include hydrophobic material to prevent fluid from entering the pump 272. The pump inlet protection mechanism 280 (or any of the inlet protection mechanisms disclosed herein) can include a filter.

The upper surface of the electronics unit 267 can include one or more indicators 266 for indicating a condition of the pump and/or level of pressure within the dressing. The indicators can be small LED lights or other light source that are visible through the dressing components or through holes in the dressing components above the indicators. The indicators can be green, yellow, red, orange, or any other color. For example, there can be two lights, one green light and one orange light. The green light can indicate the device is working properly and the orange light can indicate that there is some issue with the pump (such as, leak, saturation level of the dressing, blockage downstream of the pump, exhaust blockage, low battery, or the like).

The power source 268 can be in electrical communication with the circuit board 276. One or more power source connections are connected to a surface of the circuit board 276. The circuit board 276 can have other electronics incorporated within. For example, the circuit board 276 may support various sensors including, but not limited to, one or more pressure sensors, temperature sensors, optic sensors and/or cameras, and/or saturation indicators.

FIG. 3 illustrates an electronics assembly 300 enclosing an electronics unit within a housing. As illustrated in FIG. 3, the housing of the electronics assembly 300 can include a plate 301 and flexible film 302 enclosing the electronics unit 303 within. The electronics unit 303 can include a pump 305, inlet protection mechanism 310, pump exhaust mechanism 306, power source 307, and circuit board 309. The circuit board 309 can be flexible or substantially flexible.

As is illustrated, the pump exhaust mechanism 306 can be an enclosure, such as a chamber. The electronics unit 303 and pump 305 can be used without the inlet protection mechanism 310. However, the pump exhaust mechanism 306 and the pump 305 can sit within an extended casing 316.

The flexible film 302 can be attached to the plate 301 to form a fluid tight seal and enclosure around the electronic components. The flexible film 302 can be attached to the plate at a perimeter of the plate by heat welding, adhesive bonding, ultrasonic welding, RF welding, or any other attachment or bonding technique.

The flexible film 302 can include an aperture 311. The aperture 311 can allow the inlet protection mechanism 310 to be in fluid communication with the absorbent and/or transmission layers of the wound dressing. The perimeter of the aperture 311 of the flexible film 303 can be sealed or attached to the inlet protection mechanism 310 to form a fluid tight seal and enclosure around the inlet protection mechanism 310 allowing the electronic components 303 to remain protected from fluid within the dressing. The flexible film 302 can be attached to the inlet protection mechanism 310 at a perimeter of the inlet protection mechanism 310 by heat welding, adhesive bonding, ultrasonic welding, RF welding, or any other attachment or bonding technique. The inlet protection mechanism 310 can prevent wound exudate or liquids from the wound and collected in the absorbent area 160 of the wound dressing from entering the pump and/or electronic components of the electronics assembly 300.

The electronics assembly 300 illustrated in FIG. 3 can be incorporated within the wound dressing such that, once the dressing is applied to the body of the patient, air from within the dressing can pass through the inlet protection mechanism 310 to be pumped out toward the pump exhaust mechanism 306 in communication with an aperture in the casing 316 and the circuit board 309 as described herein.

FIGS. 4A-B illustrate an electronics assembly 400 including a pump inlet protection mechanism 410 sealed to the exterior of the flexible film 402, similar to the description with reference to FIG. 3. Also shown is an exhaust mechanism 406, which can be similar to the exhaust mechanism 306.

FIG. 4A illustrates lower, wound facing surface of the electronics assembly 400. FIG. 4B shows an upper surface of the plate 401 (which can face the patient or user) of the electronics assembly 400. The upper surface of the plate 401 can include an on/off switch or button cover 443 (illustrated as a pull tab), indicators 444, and/or one or more vent holes 442. Removal of the pull tab 443 can cause activation of the electronics assembly 400, such as provision of power from the power source to the electronics assembly. Further details of operation of the pull tab 443 are described in PCT International Application No. PCT/EP2018/079745, filed Oct. 30, 2018, titled “SAFE OPERTATION OF INTEGRATED NEGATIVE PRESSURE WOUND TREATMENT APPARATUSES,” which is incorporated by reference in its entirety herein.

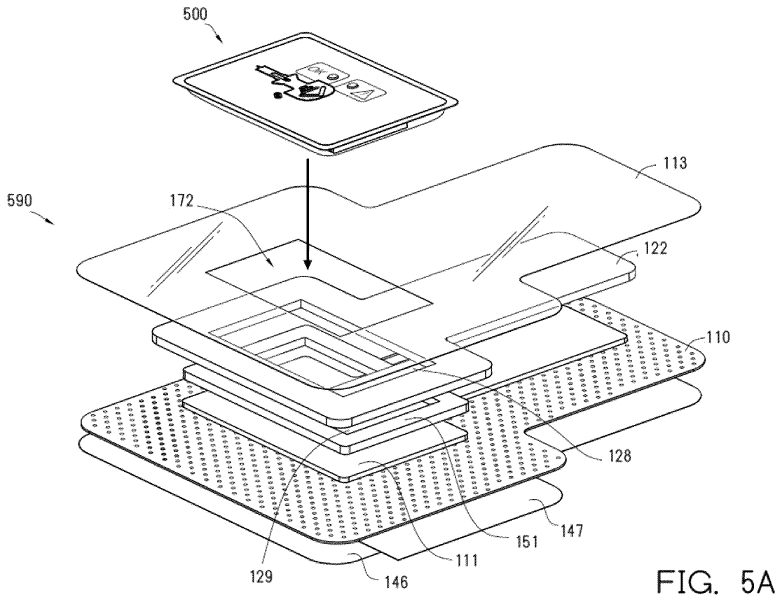

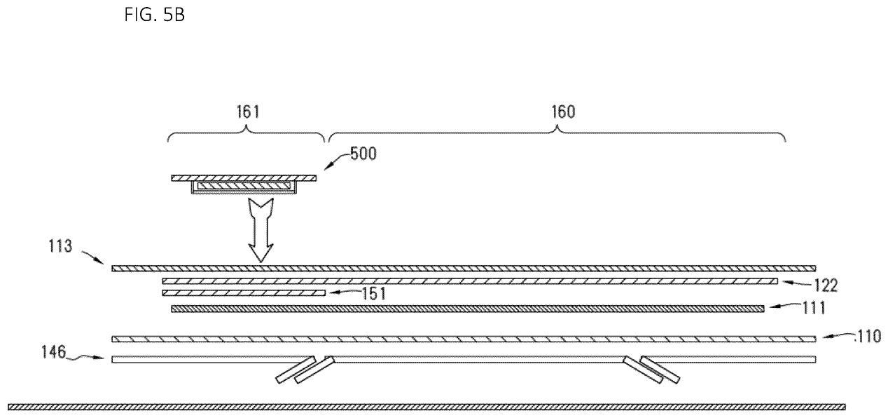

The electronics assembly 400 with the pump inlet protection mechanism 410 extending from and sealed to the film 402 can be positioned within the aperture 172 in the cover layer 113 and absorbent layer(s) (122, 151) as shown in FIG. 1C. The perimeter of the electronics assembly 400 can be sealed to a top surface of the outer perimeter of the aperture 172 in the cover layer 113 as shown in FIG. 1C and described in more detail with reference to FIG. 5A-5B herein. The electronics assembly 400 can be sealed to the cover layer 113 with a sealant gasket, adhesive, heat welding, adhesive bonding, ultrasonic welding, RF welding, or any other attachment or bonding technique. The electronics assembly 400 can be permanently sealed to the cover layer 113 and could not be removed from the cover layer without destroying the dressing.

The electronics assembly 400 can be utilized in a single dressing and disposed of with the dressing. In some cases, the electronics assembly 400 can be utilized in a series of dressings.

FIG. 5A illustrates a wound dressing, such as the one in FIG. 1C, incorporating an electronics assembly 500 within the wound dressing layers 590. FIG. 5B illustrates a cross-sectional view of the wound dressing incorporating the electronics assembly of FIG. 5A. The electronics assembly 500 can be provided within the aperture 172 in the cover layer and apertures 129 and 128 in the first and second absorbent layers 122, 151. The electronics assembly 500 can seal to the outer perimeter of the aperture 172 of the cover layer. The dressing can comprise a wound contact layer 110 and a moisture vapor permeable film, cover layer or backing layer 113 positioned above the contact layer 110 and other layers of the dressing. A layer 111 of porous material can be located above the wound contact layer 110. As used herein, the terms porous material, spacer, and/or transmission layer can be used interchangeably to refer to the layer of material in the dressing configured to distribute negative pressure throughout the wound area. This porous layer, or transmission layer, 111 allows transmission of fluid including liquid and gas away from a wound site into upper layers of the wound dressing. Further, one or more absorbent layers (such as layers 122, 151) for absorbing and retaining exudate aspirated from the wound can be utilized. The one or more layers 122, 151 of absorbent material may be provided above the transmission layer 111. There may be a small aperatured absorbent layer 151 and a large aperture absorbent layer 122. The small apertured absorbent layer 151 can be positioned on top of the large apertured absorbent layer 122. In some cases, the small apertured absorbent layer 151 can be positioned below of the large apertured absorbent layer 122. Before use, the dressing can include one or more delivery layers 146 adhered to the bottom surface of the wound contact layer. The delivery layer 146 can cover adhesive or apertures on the bottom surface of the wound contact layer 110.

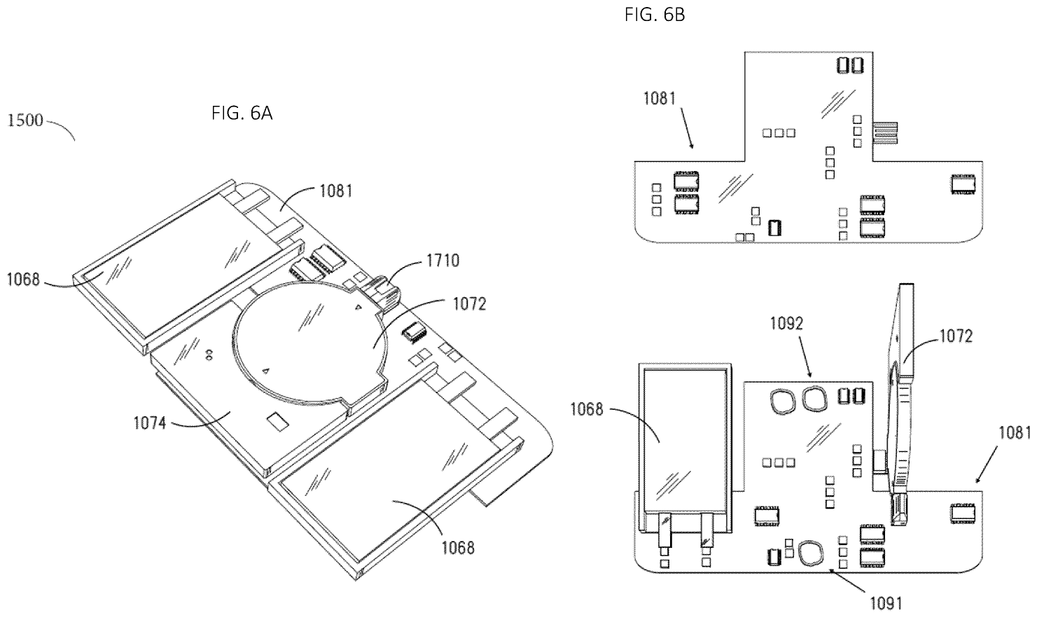

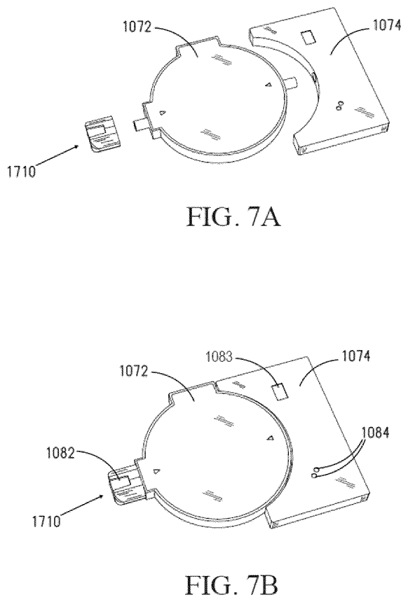

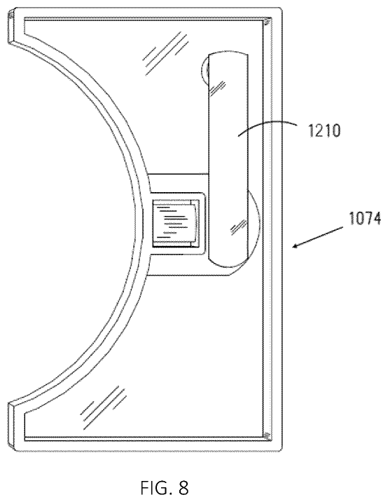

FIGS. 6A-6B and 7A-7B illustrate an electronics assembly 1500 with a pump inlet protection mechanism 1710 and pump exhaust mechanism 1074 on a pump 1072 (which can be similar to the pump 272). The assembly 1500 can include cavities 1082 and 1083 (shown in FIGS. 7A-7B) on the pump inlet protection mechanism 1710 and pump exhaust mechanism 1074, respectively. The inlet protection and pump exhaust mechanisms can be adhered to the inlet and the outlet of the pump as described herein. The assembly 1500 can be assembled using an adhesive and allowed to cure prior to incorporating into the electronics assembly.

The pump inlet can be covered or fitted with a pump inlet protection mechanism 1710. The pump inlet protection 1710 can be pushed onto the pump inlet as illustrated by the arrows in FIG. 7A. This can be a friction fit. The port of the pump inlet protection 1710 that receives a portion of the pump inlet can be sized and shaped to be a complementary fit around the pump inlet. The pump inlet protection 1710 can be bonded onto the pump inlet using a silicone sealant or any other sealant or sealing technique. FIG. 7B illustrates the pump inlet protection mechanism 1710 covering the pump inlet and the pump exhaust mechanism 1074 covering the pump outlet. The pump exhaust mechanism 1074 can include one or more apertures or vents 1084 to allow gas aspirated by the pump to be exhausted from the pump exhaust mechanism 1074. In some cases, a non-return valve and/or filter membrane of the pump exhaust mechanism is included in the pump exhaust mechanism 1074.

FIGS. 7A-7B illustrate the pump inlet protection mechanism 1710 and pump exhaust mechanism 1074 with cavities 1082 and 1083. A pump assembly including the pump inlet protection mechanism 1710 and pump exhaust mechanism 1074 can be placed over the surface of a circuit board 1081. When the pump assembly is in contact with the surface of the circuit board 1081, the cavities 1082 and 1083 can at least partially enclose sensors on the circuit board 1081, for example, pressure sensors 1091 and 1092 on the circuit board 1081, as illustrated in FIG. 6B.

The pressure sensors 1091 and 1902 illustrated in FIG. 6B can be used to measure and/or monitor the pressure level at the wound and atmospheric pressure. The pressure sensor 1091 can be used to measure and/or monitor pressure at the wound (such as, underneath the wound dressing), which can be accomplished by measuring and/or monitoring pressure in a fluid flow path connecting the negative pressure source or pump 1072 and the wound. The pressure sensor 1091 can measure and/or monitor pressure in the cavity 1082 of the pump inlet protection mechanism 1710 shown in FIGS. 7A-7B. A power source 1068 (illustrated as two batteries in FIG. 6A) can provide power to the negative pressure source 1072 and the electronics.

The pressure sensor 1092 can be used to measure and/or monitor pressure external to the wound dressing. The pressure sensor 1092 can measure and/or monitor pressure in the cavity 1083 of the pump exhaust mechanism 1074 shown in FIGS. 7A-7B. The pressure sensor 1092 can measure pressure external to the wound dressing, which can be relative atmospheric pressure since the atmospheric pressure varies depending on, for instance, an altitude of use or pressurized environment in which the TNP apparatus may be used. These measurements can be used to establish a desired negative pressure differential (or set point) at the wound relative to the external pressure.

The circuit board 1081 (including any of the circuit boards described herein) can include control circuitry, such as one or more processors or controllers, that can control the supply of negative pressure by the negative pressure source 1072 according at least to a comparison between the pressure monitored by the pressure sensor 1091 and the pressure monitored by the pressure sensor 1092. Control circuitry can operate the negative pressure source 1072 in a first mode (that can be referred to as an initial pump down mode) in which the negative pressure source 1072 is activated to establish the negative pressure set point at the wound. The set point can be set to, for example, a value in the range between about −70 mmHg to about −90 mmHg, among others. Once the set point has been established, which can be verified based on a difference between pressure measured by the pressure sensor 1091 (or wound pressure) and pressure measured by the pressure sensor 1092 (or external pressure), control circuitry can deactivate (or pause) operation of the negative pressure source 1072. Control circuitry can operate the negative pressure source 1072 is a second mode (that can be referred to as maintenance pump down mode) in which the negative pressure source 1072 is periodically activated to re-establish the negative pressure set point when the wound is depressurized as a result of one or more leaks. Control circuitry can activate the negative pressure source 1072 in response to the pressure at the wound (as monitored by the pressure sensor 1091) becomes more positive than a negative pressure threshold, which can be set to the same negative pressure as the set point or lower negative pressure.

Embodiments of the wound dressings, wound treatment apparatuses and methods described herein may also be used in combination or in addition to one or more features described in PCT International Application No. PCT/EP2017/060464, filed May 3, 2017, titled NEGATIVE PRESSURE WOUND THERAPY DEVICE ACTIVATION AND CONTROL, U.S. Pat. Nos. 8,734,425, and 8,905,985, each of which is hereby incorporated by reference in its entirety herein.

One or more self-adhesive gaskets can be applied to the pump inlet protection mechanism 1710 and pump exhaust mechanism 1074 to seal the cavities 1082 and 1083 of the pump inlet and pump exhaust around sensors on the circuit board 1081 and to seal around the exhaust mechanism vent(s) and corresponding vent(s) in the circuit board 1081 (as described herein). A pre-formed adhesive sheet can be used to form the sealing gaskets between the cavities 1082 and 1083 of the pump inlet and pump exhaust mechanisms and sensors on the circuit board 1081 and between the exhaust mechanism vent(s) and vent(s) in the circuit board 1081. In some cases, an adhesive can be used to seal the cavities 1082 and 1083 of the pump inlet protection 1710 and pump exhaust mechanism 1074 around sensors on the circuit board 1081 and to seal around the exhaust mechanism vent(s) 1084 and corresponding vent(s) in the circuit board. As described herein, the electronics assembly 1500 can be embedded within layers of the dressing, such as in cutouts or recesses into which the electronics assembly can be placed.

The pump inlet protection mechanism 1710 can provide a large surface area available for vacuum to be drawn by the inlet of the pump. A pump inlet (shown as rounded protrusion in FIG. 7A) can fit within a recess in the pump inlet protection mechanism 1710. The pump inlet can be attached by friction fit and/or form a complementary fit to the recess of the pump inlet protection mechanism.

The pump inlet protection mechanism 1710 can allow air or gas to pass through, but can block liquid from reaching the negative pressure source. The pump inlet protection mechanism 1710 can include a porous material. The pump inlet protection mechanism 1710 can comprise one or more porous polymer molded components. The pump inlet protection mechanism 1710 can include hydrophobic or substantially hydrophobic material. Material included in the pump inlet protection mechanism 1710 can have a pore size in the range of approximately 5 microns to approximately 40 microns. In some cases, the pore size can be approximately 10 microns. The pump inlet protection mechanism 1710 can include a polymer that can be one of hydrophobic polyethylene or hydrophobic polypropylene. In some cases, the pump inlet protection mechanism can include a Porvair Vyon material with a pore size of 10 microns. Any of the pump inlet protection mechanism described herein can include one or more features of the pump inlet protection mechanism 1710.

The pump exhaust mechanism 1074 (or any of the pump exhaust or outlet mechanisms described herein) can include a check valve or a non-return valve 1210 as shown in FIG. 8. The non-return valve 1210 can be any suitable mechanical one-way valve, such as, for example, a reed valve, a duckbill valve, a ball valve, a loose leaf valve or an umbrella valve, among others. The non-return valve can be similar to any of the non-return valves described in PCT International Application No. PCT/EP2017/055225, filed Mar. 6, 2017, titled WOUND TREATMENT APPARATUSES AND METHODS WITH NEGATIVE PRESSURE SOURCE INTEGRATED INTO WOUND DRESSING, which is incorporated by reference herein in its entirety. The pump exhaust mechanism 1074 can be bonded to the outlet of the pump using a sealant, for example a silicone sealant. The outlet or exhaust of the pump exhaust mechanism 1074 can include an antimicrobial film and/or other filter membrane that filters gas exhausted outside the NPWT system, such as to the atmosphere. As illustrated, pump exhaust mechanism 1074 can be an enclosure or chamber that is substantially sealed to prevent ingress of gas or fluid other than through the vent(s) 1084.

Any of the embodiments described herein can additionally or alternatively include one or more features described in International Application No. PCT/EP2018/074694, filed Sep. 13, 2018, titled NEGATIVE PRESSURE WOUND TREATMENT APPARATUSES AND METHODS WITH INTEGRATED ELECTRONICS, International Application No. PCT/EP2018/074701, filed Sep. 13, 2018, titled NEGATIVE PRESSURE WOUND TREATMENT APPARATUSES AND METHODS WITH INTEGRATED ELECTRONICS, International Application No. PCT/EP2018/079345, filed Oct. 25, 2018, titled NEGATIVE PRESSURE WOUND TREATMENT APPARATUSES AND METHODS WITH INTEGRATED ELECTRONICS, International Application No. PCT/EP2020/056317, filed Mar. 10, 2020, titled EXHAUST BLOCKAGE DETECTION FOR NEGATIVE PRESSURE WOUND TREATMENT APPARATUSES, each of which is incorporated by reference herein in its entirety.

### Soft-Start Mechanism

Any of the electronics units or assemblies disclosed herein (such as, the electronics unit 267 or the electronics assembly 1500) can include soft-start control circuitry configured to limit current draw from a power source (such as, the power source 268 or 1068) and prevent a brownout condition from occurring that would otherwise reset or disable any of the components of the electronics unit or assembly. Under certain operating conditions, an inrush current (which can be associated with a large current that rushes into a circuit at the initial stage) can cause the power source to become unable to maintain a stable voltage (or current). In these circumstances the voltage (or current) supplied by the power source can drop substantially below the nominal operating voltage of the electronics unit or assembly, causing a brownout condition. In turn, this can undesirably cause a reset of one or more components of the electronics unit or assembly, such as one or more digital electronic components (for instance, a controller). For example, to mitigate uncertain behavior caused by brownouts, a microcontroller can be configured to automatically reset when a supply voltage drops below an operating voltage threshold (such as, 3.3 V or 5 V). A brownout and subsequent reset can cause interruption of negative pressure wound therapy. It is preferable to avoid brownouts through consistent power delivery.

A soft-start mechanism described herein can be incorporated into the electronics unit or assembly to reduce the initial current drawn from the power source and prevent occurrence of a brownout condition. The soft-start mechanism can be implemented in software or firmware. Advantageously, little additional hardware or no additional hardware may be needed and little or no modifications to the existing circuit design may be needed. Static or dynamic optimization can be used to mitigate various factors (such as, component tolerance, parasitic characteristics, environmental changes, or the like) to reduce (or eliminate) brownouts.

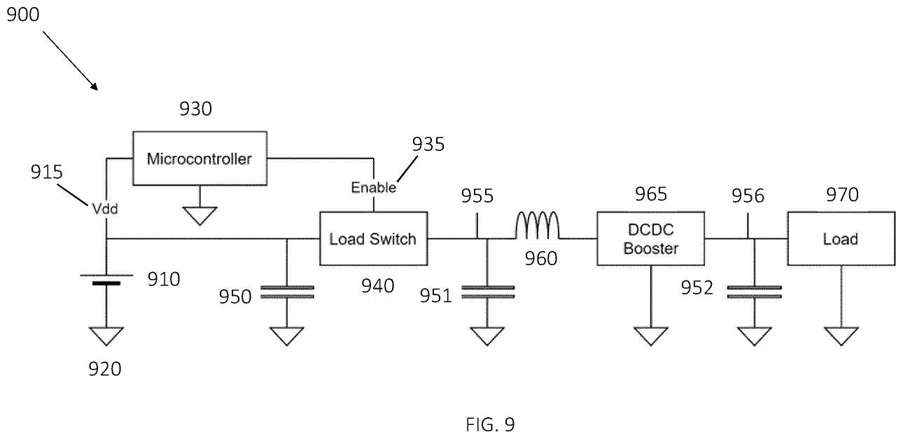

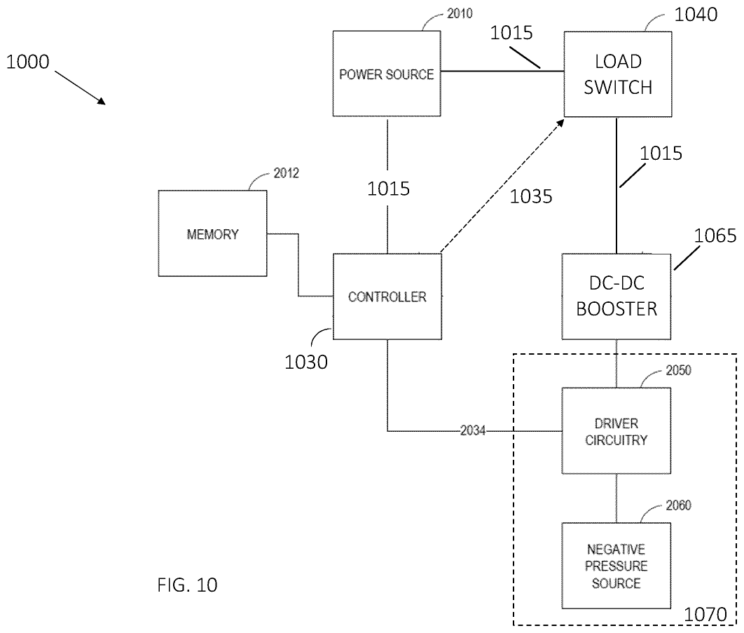

Referring now to FIG. 9, a soft-start control circuitry 900 is illustrated. The control circuitry 900 includes the power source 910, which can be one or more batteries. The power source 910 can include one or more coin cell batteries, such as a CR2032 or CR2025. The power source 910 can include any other source of power with high resistance connected in series to the power source (such as, one or more wires, conductive ink, or the like). The power source 910 can supply a voltage 915 (Vdd) to the other electronic components, including a controller 930 (which can be a microcontroller) and a switch 940 (sometimes referred to as a load enable switch). Normally, the voltage 915 is a nominal voltage of the power source 910 (such as, the nominal battery voltage) . . . . In some cases, the nominal voltage 915 is in a range of about 2 to 3 volts, but may be expected to vary over the life cycle of the power source 910. The controller 930 can provide an enable signal 935 to the switch 640, which causes the switch 940 to be activated, allowing current to pass. The enable signal can be pulsed (or switched) between active (on) and inactive (off) states to control the activation and deactivation of the switch 940. The control circuitry 900 can include a first capacitor 950 and second capacitor 951. The first capacitor 950 can be connected between a first terminal of the switch 940 and the ground 920. The second capacitor 951 can be connected between a second terminal of the switch 940 and the ground 920, and its operation is described below. The first capacitor 950 can be configured to charge up to the nominal voltage (or another voltage level) from the power source 910 when the switch 940 is deactivated. The first capacitor 950 can be configured to provide additional power (for instance, in addition to the voltage provided by the power source 910).

The second terminal of the switch 940 is electrically connected to a power converter 965, which can be a boost converter. The boost converter 965 can convert the power provided from the power source 910 to a level suitable for driving a load 970 (such as, a negative pressure source). In some cases, the boost converter 965 is a DC-DC (direct current-direct current) converter.

As described herein, the second terminal can serve as a first test point 955 for voltage measurements by the controller 930. The controller 930 can be configured to receive voltage measurement data from the first test point 955 and utilize the measurements to control the enable signal 935 in order to activate or deactivate the switch 935. An inductor 960 can connect the second terminal of the switch 940 to the boost converter 965. In certain cases, the inductor 960 and the second capacitor 951 can form an LC (inductor-capacitor) circuit, which can operate as a bandpass filter. The LC circuit can resonate at a resonance frequency (which can be equal to

$$\frac{1}{2 π √ LC}$$

where L is the inductance of the inductor 960 and C is the capacitance of the second capacitor 951).

On initiation (or power up), the first capacitor 950 can be charged by the power source 910. At this time, the switch 940 can be maintained in the inactive (or disabled) state. When the controller 930 activates the enable signal 935 causing the switch 940 to be active, current can be drawn from the power source 910 (and/or the first capacitor 950) to charge the second capacitor 951, inductor 960, and supply power to the boost converter 965, which in turn provides power to charge a third capacitor 952. The boost converter 965 can step up the voltage 915 provided by the power supply 910 to supply a boosted voltage to the load 970. In some cases, the boosted voltage output by the booster converter 965 is approximately 3.6 V, whereas the nominal voltage is between approximately 2 V and 3 V. In certain cases, the boost converter 965 is configured to supply an output voltage greater than 3.6 V to meet a higher power requirement of the load 970. Depending on the power source 910 (such as, battery type, age of the batteries, and battery configuration), the nominal voltage may be lower than 2 V or higher than 3 V. The third capacitor 952 can be provided and connected between the output of the boost converter 965 and the ground 920. The third capacitor 952 can be configured to provide additional power to the load 970 (for instance, in addition to the stepped-up voltage provided by the power source boost converter 965).

When the switch 940 is active, current drawn from the power source 910 (and/or the first capacitor 950) can charge the second and third capacitors 951 and 952 as well as the inductor 960. A large inrush current can be drawn to charge these electronic components. If the first capacitor 950 is insufficient (or not present), the additional power would need to come from the power source 910. In turn, the inrush current can result in a significant voltage drop across the power source 910 (which can include one or more batteries), which can lead to a brownout and/or reset of the controller 930.

To mitigate or prevent the brownout and/or reset of the controller 930, the inrush current can be limited. This can be accomplished by pulsing the enable signal 935 and, as a result, alternating periods of activation of the switch 940. In order to avoid the inrush current, the controller 930 can alternate active and inactive portions of the enable signal 935. As a result, transfer of energy from the power source 910 can be packetized by utilizing and controlling the switch 940. The maximum duration of the active portion of the enable signal 935 can be constrained by the acceptable voltage drop across the power source 910, which can be associated with the maximum allowable voltage drop that would not trigger a brownout (and a reset of the controller 930). The maximum allowable voltage drop may be calculated to include a safety voltage buffer, such as at least 0.2 V (or less or more), to prevent an accidental reset of the controller 930. The controller 930 can be configured to receive voltage measurement data from the first test point 955 and calculate a difference between the instantaneous voltage measured from the power source 910 and the minimum voltage required by the controller. The controller 930 can utilize the following formula to calculate the maximum acceptable voltage drop (V_{drop(max)}), where Vs is the instantaneous voltage of the power source 910, Vₘᵢₙ is the minimum voltage required by the controller 930, and V_{buffer} is the safety voltage buffer.

$${V}_{drop ( max )} = {V}_{source} - ( {V}_{min} + {V}_{buffer} )$$

For instance, in some cases, the controller requires a supply voltage of at least 1.8 V to avoid a brownout condition and reset. Using a safety buffer of 0.2 V, the maximum acceptable voltage drop at 2.5 V can be determined as 0.5 V (2.5 V−(1.8 V+0.2 V)).

The minimum duration of the inactive portion of the enable signal 935 can be constrained by the recovery time of the power source 910, which can be associated with the duration of time needed to restore the voltage of the power source 910 to the nominal voltage 915. Recovery time can be related to the previous inrush current spikes, and advantageously, reducing the inrush current can reduce the required recovery time. Recovery time can be measured in use, for instance, by monitoring the voltage across the power source 910 (for instance, by the controller 930 monitoring the voltage supplied by the power source 910 to the controller).

One or more of the frequency or duty cycle of the enable signal 935 can be varied. In some cases, the frequency of pulsing the enable signal 935 between active and inactive states can be equal to (or approximately equal to) the resonance frequency of the LC circuit formed by the second capacitor 951 and the inductor 960. The controller 930 can pulse the switch 940 by generating the enable signal 935 at brief, regular intervals. Pulse width modulation (PWM) can be used to generate the enable signal 935 for controlling the switch 940. The controller 930 can be configured to send a predetermined number of pulses of the enable signal 935 at a predetermined duty cycle and/or frequency. In some cases, the enable signal 935 can include 50 pulses at a frequency of about 277 kHz and about 50% duty cycle. In certain implementations, the enable signal 935 can include 20 pulses at a frequency of 10 kHz and about 1% duty cycle.

The controller 930 can utilize closed loop control, such as a proportional-integral-derivative control loop (PID) or proportional-integral control loop (PI), to generate the enable signal 935. The controller 930 can monitor the voltage difference between the first test point 955 and a second test point 956 and utilize the voltage difference as feedback from the control loop. The control loop can control one or more PWM parameters (such as, duty cycle and/or frequency) to generate the enable signal 935. For example, the duty cycle of PWM signal can be increased over time (such as, from 10/90 (10% on, 90% off), to 20/80, to 30/70, to 40/60, to 50/50 and so on to 90/10 and finally to 100/0). Closed loop control can provide better management of the inrush current.

The controller 930 can use threshold detection to control the switch 940. By monitoring the voltage 915 received from the power source 2010 during operation, the controller 930 can deactivate (or disable) the switch 940 responsive to the voltage 915 satisfying (such as, dropping below) a first voltage threshold. The first voltage threshold can be selected as the maximum allowable voltage drop that would not trigger a brownout, as described herein. The controller 930 can activate the switch 940 responsive to the voltage 915 satisfying a second voltage threshold or a timeout period. The second voltage threshold can correspond to sufficient level of voltage such that a brownout would not occur. The second voltage threshold can correspond to higher voltage than the first voltage threshold. The timeout period can be selected as the recovery time, as described herein. In some cases, the second voltage threshold and/or timeout period can be selected to avoid current draw from the power source 910 exceeding a maximum current draw recommended by the manufacturer.

Pulsing of the enable signal 940 to activate and deactivate the switch 940 can be performed until the energy storage components (such as, the second capacitor 951, inductor 960, and the third capacitor 952) are sufficiently charged. When this occurs, the risk of a voltage drop across the power source 910 that would cause a brownout can be eliminated. In some cases, pulsing of the enable signal 935 can be stopped after a duration of time, which can be representative of the amount of time needed to sufficiently charge the energy storage components.

As described herein, the switch 940 can be a transistor (such as, a bipolar junction transistor or a field-effect transistor). The controller 930 can generate the enable signal 935 to operate the transistor in the linear (or active) region. By operating the transistor in the linear region, the output current of the transistor can be limited (for instance, limited relative to the voltage of the enable signal 935). The overall efficiency of the power delivery to the load 970 can be improved by maintain the transistor in the linear region.

While certain implementations describe enabling and disabling the switch 940, the power converter 965 can include an enable line (or pin) which can be enabled or disabled as described herein.

FIG. 10 illustrates a block diagram of an electronics assembly 1000 of a TNP system (which can be any of the TNP systems described herein). The electronics assembly 1000 can include similar components as the control circuitry 900 in FIG. 9. Similar components can be designated in FIG. 10 as “10XX” or “20XX” instead of 9XX in FIG. 9. The electronics assembly can include a power source 2010, a controller 1030, a switch 1040, a memory 2012, a boost converter 1065, driver circuitry 2050, and a negative pressure source 2060. Collectively, the driver circuitry 2050 and the negative pressure source 2060 can form a load 1070. The negative pressure source 2060 can include a piezoelectric pump (such as, a pump operated by a piezoelectric actuator or transducer). In some cases, the driver circuitry 2050 can include H-bridge circuitry. The power source 2010 may include one or more batteries (such as, two 3V batteries). The power source 2010 may provide power to the other electronics components.

As described herein, the power source 2010 can provide voltage 1015 to the controller 1030 and the switch 1040. The controller 1030 can generate an enable signal 1035 to control the switch 1040.

Power supplied by the power source 2010 (such as, 3V or less or more, 4V or less or more, 5V or less or more, 6V or less or more, 7V or less or more, 8V or less or more, 9V or less or more, or the like) may need to be increased for powering the negative pressure source 2060. The boost converter 965 can increase the power provided by the power source 2010 to a power level adequate for powering the negative pressure source 2060. The boost converter 1065 can include electronic circuitry configured to generate a higher level of power (for example, higher voltage of DC power) from a lower input power (for example, battery power). In some cases, the boost converter 1065 can be a switched-mode power supply. The boost converter 1065 can be a DC to DC converter with an output voltage greater than the input or source voltage. The boost converter 1065 can increase or step up the power level of the power source 2010 to a power level for operating the negative pressure source 2060. For example, the power source 2010 may provide 3V DC power (or less or more) and the negative pressure source 2060 can require DC power between 30V (or less or more) and 22V (or less or more).

The controller 1030 can operate the driver circuitry 2050 via a signal 2034 (such, as a pulse width modulation signal). The negative pressure source 2060 can be controlled by alternating periods of activation and deactivation of the negative pressure source. A duty cycle of the negative pressure source 2060 can reflect a portion of time during which the negative pressure source is active relative to a given time interval (such as, 1 second, 5 seconds, 10 seconds, 20 seconds, 30 seconds, etc.). For example, if the negative pressure source 2060 is active for 15 seconds over a 30 second time interval, the duty cycle is 50%. In some cases, increase in the duty cycle of the negative pressure source 2060 can be indicative or a presence of one or more leaks (for example, in the seal between the dressing and the patient). This may be due to the negative pressure source 2060 being active longer in order to establish or maintain a negative pressure set point in presence of one or more leaks. Further details of operation of the boost converter and operation of the negative pressure source are described in International Application No. PCT/EP2020/064601, filed Apr. 26, 2020, titled “SYSTEMS AND METHODS FOR EXTENDING OPERATIONAL TIME OF NEGATIVE PRESSURE WOUND TREATMENT APPARATUSES” and UK Application No. GB 2104922.6, filed on Apr. 7, 2021, titled “TEMPERATURE MONITORING AND CONTROL FOR NEGATIVE PRESSURE WOUND THERAPY SYSTEMS,” each of which is incorporated by reference in its entirety herein.

### User Activity Monitoring

Any of the soft-start mechanisms described herein can be integrated into a patient monitoring and/or treatment device. Activities of a user may be desirably monitored by an activity monitoring device for a variety of reasons including wound prevention and monitoring. In one example, the activities of a user can be monitored when the user may be prone to or already have a pressure ulcer. Information gathered by the activity monitoring device about the activities of the user can be helpful for assisting with prevention or treatment of the pressure ulcer. In addition, information gathered by the activity monitoring device about the activities can be useful for checking compliance with a treatment regimen.

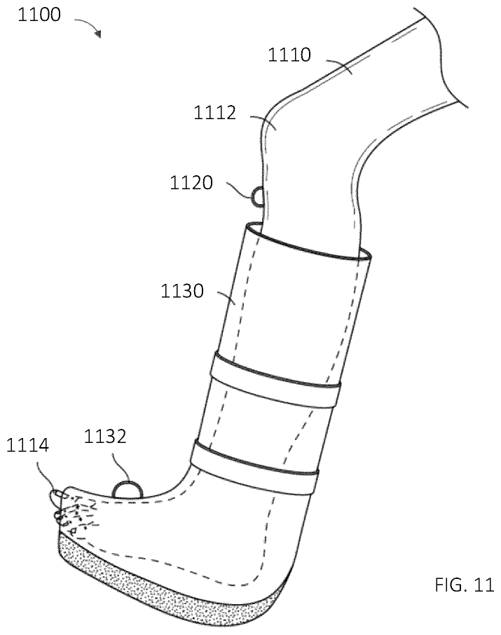

FIG. 11 illustrates a user activity monitoring system 1100 including an activity monitoring device 1120 attached to a body part 1110. The activity monitoring device 1120 can be attached to the body part 1110 using a strap, adhesive, or other coupling mechanism and may be worn on or supported by the body.

The body part 1110 can be a leg of a user that includes a knee 1112 and a foot 1114. As illustrated, in some embodiments, the activity monitoring device 1120 can be supported by the body part 1110 at a position between the knee 1112 and the foot 1114, such as proximate to the foot 1114. In other embodiments, the activity monitoring device 1120 can be supported by another part of the body part 1110. The activity monitoring device 1120 can monitor and record activities (for instance, walking, jumping, sitting, laying down, running, squatting, or standing) of the body part 1110, such as from a position, movement, or orientation of the activity monitoring device 1120 or one or more other sensors of the activity monitoring device 1120. The activity monitoring device 1120 can, for example, be used for loading monitoring of loading of the foot 1114. In certain implementations, multiple body parts can be monitored by the activity monitoring device 1120, and different sensors can be used for monitoring different body parts.

The body part 1110 is shown wearing and partly covered by an orthopedic device 1130. The orthopedic device 1130 can support the body part 1110 and reduce a pressure on the foot 1114 when the user may be standing or engaging in other activities. A compliance monitoring device 1132 can be attached to the orthopedic device 1130. The compliance monitoring device 1132 can be the same as or different from the activity monitoring device 1120 and supported by the orthopedic device 1130 using a strap, adhesive, or other coupling mechanism. The compliance monitoring device 1132 can be attached to an inner surface of the orthopedic device 1130 such that the compliance monitoring device 1132 is disposed between the orthopedic device 1130 and the skin of the patient. The compliance monitoring device 1132 can be attached to an outer surface of the orthopedic device 1130 such that a portion of the orthopedic device 1130 is disposed between the compliance monitoring device 1132 and the skin of the patient. Although not shown in FIG. 11, the compliance monitoring device 1132 can be attached to an orthopedic device 1130 that is not worn by the patient (for example, a cane, a walker).

Although not illustrated in FIG. 11, the user activity monitoring system 1100 can additionally or alternatively include one or more of the activity monitoring device 1120 or the compliance monitoring device 1132 at other positions, such as at a position supported by the orthopedic device 1130 or another part of the body part 1110. These one or more additional or alternative of the activity monitoring device 1120 or the compliance monitoring device 1132 can be the same as or similar to the activity monitoring device 1120 may monitor and record activities of the orthopedic device 1130 or the another part of the body part 1110.

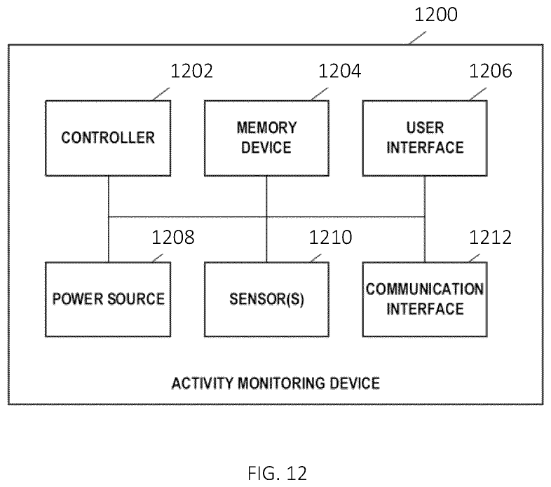

FIG. 12 illustrates a schematic 1200 of an activity monitoring device, such as the activity monitoring device 1120. The activity monitoring device and the data processing device can together configure a communication channel with one another to permit transfer of recorded activities or other data from the activity monitoring device and the data processing device or transfer of one or more commands from the data processing device to the activity monitoring device, among other possibilities. The data processing device can, for example, be a smart phone or a tablet computer.

As illustrated by the schematic 1200 in FIG. 12, the activity monitoring device can include a controller 1202, a memory device 1204, a user interface 1206, a power source 1208, one or more sensors 1210, and a communication interface 1212 that are configured to electrically communicate with one another. The power source 1208 can provide power to one or more components of the activity monitoring device. The components of the activity monitoring device can be contained in or supported by a housing of the activity monitoring device as discussed in more detail below.

The controller 1202 can control operations of one or more other components of the activity monitoring device according at least to instructions stored in the memory device 1204. The controller 1202 can, for instance, control monitoring of loading of the body part 1110 with a weight of the body or positioning of the body part 1110 and record data indicative of loading of the body part 1110 or positioning of the body part 1110 to the memory device 1204.

The controller 1202 can generate an enable signal to prevent a brownout condition, as described herein. A load can include one or more of the various sensors 1210 of the monitoring device, the user interface 1206, or the communication interface 1212.

The user interface 1206 can include one or more output elements, such as indicators (for example, light emitting diodes) or speakers, that provide user outputs to a user. The one or more output elements can convey status information to the user like whether the activity monitoring device is successfully functioning or has successfully configured communication with the data processing device. The user interface 1206 can further include one or more input elements, such as buttons, switches, dials, or touch screens, for receiving user inputs for configuring the activity monitoring device. In some embodiments, the user interface 1206 may have no more than one user input element, such as a button, for receiving user inputs to activate and deactivate the activity monitoring device or performing one or more other functions.

The one or more sensors 1210 can be used to detect and monitor a motion of the activity monitoring device. The one or more sensors 1210 can be used to detect and monitor activities of the user of the activity monitoring device that include, for instance, a loading or positioning of the body part 1110. The one or more sensors 1210 can include one or more accelerometers, gyroscopes, magnetometers, pressure sensors, impedance sensors, thermistors, or optical sensors, among other types of sensors. The one or more sensors 1210 can be positioned proximate to the body part 1110 or may be remote from the body part 1110 yet usable to monitor characteristics of the body part 1110.

The communication interface 1212 can be used to communicate with the data processing device, such as via radio waves and according to a Bluetooth™ protocol like Bluetooth™ Low Energy or another protocol. The communication interface 1212 can, for example, transmit device usage data like alarms, monitored loading or positioning, or changes to a monitoring or therapy program performed by the activity monitoring device to the data processing device. The communication interface 1212 can be used to receive data, including commands, from the data processing device.

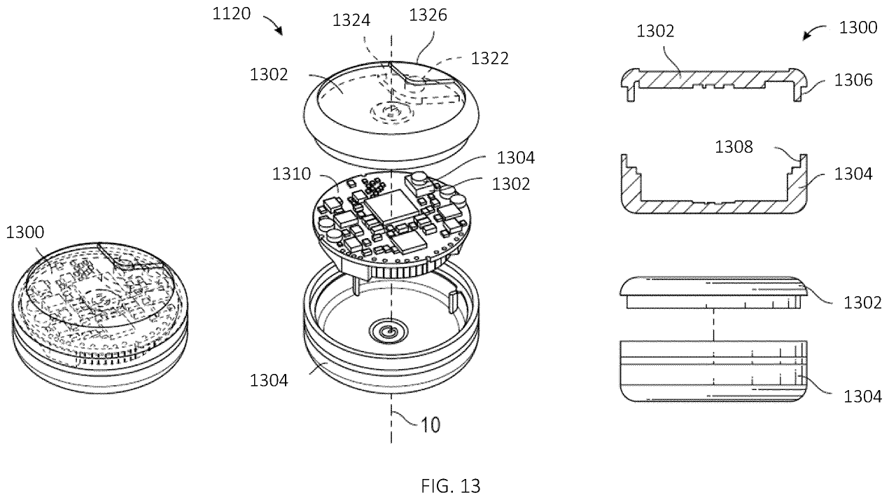

FIG. 13 illustrates an activity monitoring device, such as the activity monitoring device 1120. The housing 1300 can be arranged to enclose a printed circuit board assembly (PCBA) 1310. The housing 1300 can have a cap portion 1302 and a base portion 1304. The cap portion 1302 and the base portion 1304 can be joined together to form an enclosed space within the housing 1300. The enclosed space can be sized to at least accommodate the PCBA 1310. In the embodiment shown in FIG. 13, the cap portion 1302 has an inner rim 1306 that nests inside an outer sidewall 1308 disposed on the base portion 1304 to form the enclosed space within the housing 1300. The orientation of the inner rim 1306 and the outer sidewall 1308 can be reversed, in some implementations, such that the inner rim 1306 is disposed on the base portion 1304 and the outer sidewall 1308 is disposed on the cap portion 1302.

The PCBA 1310 can include one or more of the components of the activity monitoring device 1120 such as the controller 1202, the memory device 1204, the user interface 1206, the power source 1208, the sensor(s) 1210, and the communication interface 1212. In some embodiments, one or more of the components of the activity monitoring device 1120 can be mounted to a portion of the activity monitoring device 1120 other than the PCBA 1310, such as the housing 1300. As shown in FIG. 13, the PCBA 1310 can include a microswitch 1302. The PCBA 1310 can include more than one microswitch 1302. The microswitch 1302 can include a push button 1304. The microswitch 1302 can be activated by applying a compressive force to the push button 1304.

The housing 1300 can be arranged such that a targeted or intended compression of the housing 1300 within a particular vicinity of the microswitch 1302 activates the microswitch 1302. The housing 1300 can include features that cause the push button 1304 to be pressed when a targeted compression is applied to the housing 1300 in the vicinity of the microswitch 1302. The housing 1300 can be arranged such that an untargeted or unintentional compression of the housing 1300 outside a particular vicinity of the microswitch 1302 does not activate the microswitch 1302. The housing 1300 can be arranged such that the housing 1300 shields the PCBA 1310 from compressive forces that are applied to the housing 1300.

### Other Variations

While certain embodiments described herein relate to integrated negative pressure wound therapy systems in which the negative pressure source is supported by the dressing, systems and methods described herein are applicable to any negative pressure wound therapy system or medical system, particularly to systems being positioned on (or worn by) the patient. For example, systems and methods for controlling operation described herein can be used in fluid-proof (such as, water-proof) negative pressure wound therapy systems or medical systems. Such systems can be configured with the negative pressure source and/or electronics being external to the wound dressing, such as with the negative pressure source and/or electronics being positioned in a fluid proof enclosure. Additionally, such systems can be configured to be used within ultrasound delivery devices, negative pressure devices powered by an external power supply, negative pressure devices with a separate pump, and medical devices generally.

Any of the embodiments disclosed herein can be used with one or more features disclosed in U.S. Pat. No. 7,779,625, titled “DEVICE AND METHOD FOR WOUND THERAPY,” issued Aug. 24, 2010; U.S. Pat. No. 7,964,766, titled “WOUND CLEANSING APPARATUS IN SITU,” issued on Jun. 21, 2011; U.S. Pat. No. 8,235,955, titled “WOUND TREATMENT APPARATUS AND METHOD,” issued on Aug. 7, 2012; U.S. Pat. No. 7,753,894, titled “WOUND CLEANSING APPARATUS WITH STRESS,” issued Jul. 13, 2010; U.S. Pat. No. 8,764,732, titled “WOUND DRESSING,” issued Jul. 1, 2014; U.S. Pat. No. 8,808,274, titled “WOUND DRESSING,” issued Aug. 19, 2014; U.S. Pat. No. 9,061,095, titled “WOUND DRESSING AND METHOD OF USE,” issued Jun. 23, 2015; U.S. Pat. No. 10,076,449, issued Sep. 18, 2018, titled “WOUND DRESSING AND METHOD OF TREATMENT”; U.S. patent application Ser. No. 14/418,908, filed Jan. 30, 2015, published as U.S. Publication No. 2015/0190286, published Jul. 9, 2015, titled “WOUND DRESSING AND METHOD OF TREATMENT”; U.S. Pat. No. 10,231,878, titled “TISSUE HEALING,” issued Mar. 19, 2019; PCT International Application PCT/GB2012/000587, titled “WOUND DRESSING AND METHOD OF TREATMENT” and filed on Jul. 12, 2012; International Application No. PCT/IB2013/001469, filed May 22, 2013, titled “APPARATUSES AND METHODS FOR NEGATIVE PRESSURE WOUND THERAPY”; PCT International Application No. PCT/IB2013/002102, filed Jul. 31, 2013, titled “WOUND DRESSING AND METHOD OF TREATMENT”; PCT International Application No. PCT/IB2013/002060, filed Jul. 31, 2013, titled “WOUND DRESSING AND METHOD OF TREATMENT”; PCT International Application No. PCT/IB2013/00084, filed Mar. 12, 2013, titled “REDUCED PRESSURE APPARATUS AND METHODS”; International Application No. PCT/EP2016/059329, filed Apr. 26, 2016, titled “REDUCED PRESSURE APPARATUSES”; PCT International Application No. PCT/EP2017/059883, filed Apr. 26, 2017, titled “WOUND DRESSINGS AND METHODS OF USE WITH INTEGRATED NEGATIVE PRESSURE SOURCE HAVING A FLUID INGRESS INHIBITION COMPONENT”; PCT International Application No. PCT/EP2017/055225, filed Mar. 6, 2017, titled “WOUND TREATMENT APPARATUSES AND METHODS WITH NEGATIVE PRESSURE SOURCE INTEGRATED INTO WOUND DRESSING”; PCT International Application No. PCT/EP2018/074694, filed Sep. 13, 2018, titled “NEGATIVE PRESSURE WOUND TREATMENT APPARATUSES AND METHODS WITH INTEGRATED ELECTRONICS”; PCT International Application No. PCT/EP2018/074701, filed Sep. 13, 2018, titled “NEGATIVE PRESSURE WOUND TREATMENT APPARATUSES AND METHODS WITH INTEGRATED ELECTRONICS”; PCT International Application No. PCT/EP2018/079345, filed Oct. 25, 2018, titled “NEGATIVE PRESSURE WOUND TREATMENT APPARATUSES AND METHODS WITH INTEGRATED ELECTRONICS”; PCT International Application No. PCT/EP2018/079745, filed Oct. 30, 2018, titled “SAFE OPERTATION OF INTEGRATED NEGATIVE PRESSURE WOUND TREATMENT APPARATUSES”; each of which is incorporated by reference herein in its entirety.

Although certain embodiments described herein relate to wound dressings, systems and methods disclosed herein are not limited to wound dressings or medical applications. Systems and methods disclosed herein are generally applicable to electronic devices in general, such as electronic devices that can be worn by or applied to a user.

Any value of a threshold, limit, duration, etc. provided herein is not intended to be absolute and, thereby, can be approximate. In addition, any threshold, limit, duration, etc. provided herein can be fixed or varied either automatically or by a user. Furthermore, as is used herein relative terminology such as exceeds, greater than, less than, etc. in relation to a reference value is intended to also encompass being equal to the reference value. For example, exceeding a reference value that is positive can encompass being equal to or greater than the reference value. In addition, as is used herein relative terminology such as exceeds, greater than, less than, etc. in relation to a reference value is intended to also encompass an inverse of the disclosed relationship, such as below, less than, greater than, etc. in relations to the reference value. Moreover, although blocks of the various processes may be described in terms of determining whether a value meets or does not meet a particular threshold, the blocks can be similarly understood, for example, in terms of a value (i) being below or above a threshold or (ii) satisfying or not satisfying a threshold.

Features, materials, characteristics, or groups described in conjunction with a particular aspect, embodiment, or example are to be understood to be applicable to any other aspect, embodiment or example described herein unless incompatible therewith. All of the features disclosed in this specification (including any accompanying claims, abstract and drawings), or all of the steps of any method or process so disclosed, may be combined in any combination, except combinations where at least some of such features or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

While certain embodiments have been described, these embodiments have been presented by way of example only, and are not intended to limit the scope of protection. Indeed, the novel methods and systems described herein may be embodied in a variety of other forms. For example, while the housing has been shown to be mounted on an outer surface of the dressing, in some variants the housing and/or the electronics of the activity-monitoring device can be encapsulated in the dressing. Furthermore, various omissions, substitutions and changes in the form of the methods and systems described herein may be made. Those skilled in the art will appreciate that in some embodiments, the actual steps taken in the processes illustrated or disclosed may differ from those shown in the figures. Depending on the embodiment, certain of the steps described above may be removed, others may be added. For example, the actual steps or order of steps taken in the disclosed processes may differ from those shown in the figure.

Depending on the embodiment, certain of the steps described above may be removed, others may be added. The various components illustrated in the figures may be implemented as software or firmware on a processor, controller, ASIC, FPGA, or dedicated hardware. The software or firmware can include instructions stored in a non-transitory computer-readable memory. The instructions can be executed by a processor, controller, ASIC, FPGA, or dedicated hardware. Hardware components, such as controllers, processors, ASICs, FPGAs, and the like, can include logic circuitry. Furthermore, the features and attributes of the specific embodiments disclosed above may be combined in different ways to form additional embodiments, all of which fall within the scope of the present disclosure.

Although the present disclosure includes certain embodiments, examples and applications, it will be understood by those skilled in the art that the present disclosure extends beyond the specifically disclosed embodiments to other alternative embodiments or uses and obvious modifications and equivalents thereof, including embodiments which do not provide all of the features and advantages set forth herein. Accordingly, the scope of the present disclosure is not intended to be limited by the specific disclosures of preferred embodiments herein, and may be defined by claims as presented herein or as presented in the future.

Conditional language, such as “can,” “could,” “might,” or “may,” unless specifically stated otherwise, or otherwise understood within the context as used, is generally intended to convey that certain embodiments include, while other embodiments do not include, certain features, elements, or steps. Thus, such conditional language is not generally intended to imply that features, elements, or steps are in any way required for one or more embodiments or that one or more embodiments necessarily include logic for deciding, with or without user input or prompting, whether these features, elements, or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount.

The scope of the present disclosure is not intended to be limited by the specific disclosures of preferred embodiments in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

1. A negative pressure wound therapy system comprising:
a battery;
a power converter electrically connected to the battery and configured to increase voltage provided by the battery;
a load configured to receive power from the power converter, the load including a negative pressure source configured to provide negative pressure to a wound of a patient covered by a wound dressing;
a switch electrically connected to the battery and the power converter and positioned between the battery and the power converter; and
a control circuitry electrically connected to the switch and configured to alternate activating and deactivating the switch to reduce peak current provided by the battery, thereby preventing a brownout condition and reset of the control circuitry.

2. The negative pressure wound therapy system of claim 1, further comprising at least one energy storage component configured to be charged by the battery, wherein the control circuitry is configured to maintain the switch in an active state responsive to determining that the at least one energy storage component has been charged or responsive to an expiration of a threshold duration of time.

3. The negative pressure wound therapy system of claim 1, wherein the control circuitry is configured to generate a pulse signal to alternate activating and deactivating the switch.

4. The negative pressure wound therapy system of claim 3, wherein a first duration of time during which the pulse signal is active is determined from a threshold voltage drop across the battery, and wherein a second duration of time during which the pulse signal is inactive is determined from a recovery time of the battery.

5. The negative pressure wound therapy system of claim 4, wherein the control circuitry is further configured to vary at least one of a frequency or duty cycle of the pulse signal.

6. The negative pressure wound therapy system of claim 1, wherein the power converter comprises a DC-to-DC converter.

7. The negative pressure wound therapy system of claim 1, wherein the battery comprises a coin cell battery.

8. The negative pressure wound therapy system of claim 1, wherein the switch comprises a transistor.

9. The negative pressure wound therapy system of claim 1, wherein the control circuitry is configured to pulse the switch at a predetermined frequency and duty cycle.

10. The negative pressure wound therapy system of claim 9, wherein the predetermined frequency is associated with a resonant frequency of an LC circuit electrically connected to the power converter.

11. (canceled)

12. A patient monitoring and/or treatment device comprising:
a power source;
a power converter electrically connected to the power source and configured to increase voltage provided by the power source;
a load configured to receive power from the power converter;
a switch electrically connected to the power source and the power converter and positioned between the power source and the power converter; and
a control circuitry electrically connected to the switch and configured to alternate activating and deactivating the switch to reduce peak current provided by the power source.

13. The patient monitoring and/or treatment device of claim 12, wherein the power source comprises a coin cell battery.

14. The patient monitoring and/or treatment device of any of claim 12, wherein the control circuitry is configured to generate a pulse signal to alternate activating and deactivating the switch.

15. The patient monitoring and/or treatment device of claim 14, wherein a first duration of time during which the pulse signal is active is determined from a threshold voltage drop across the power source, and wherein a second duration of time during which the pulse signal is inactive is determined from a recovery time for the power source.

16. The patient monitoring and/or treatment device of claim 15, wherein the control circuitry is further configured to vary at least one of a frequency or duty cycle of the pulse signal.

17. The patient monitoring and/or treatment device of any of claim 12, wherein the power converter comprises a DC-to-DC converter.

18. The patient monitoring and/or treatment device of claim 12, wherein the switch comprises a transistor.

19. The patient monitoring and/or treatment device of claim 12, wherein the control circuitry is configured to pulse the switch at a predetermined frequency and duty cycle.

20. The patient monitoring and/or treatment device of claim 19, wherein the predetermined frequency is associated with a resonant frequency of an LC circuit electrically connected to the power converter.

21. (canceled)

22. A method of operating a patient monitoring and/or treatment device, the method comprising:
by a control circuitry:
activating a switch to electrically connect a power source to a load;
monitoring a voltage differential across the power source;
in response to determining that the voltage differential satisfies a maximum voltage drop threshold, deactivating the switch to disconnect the power source from the load; and
activating the switch in response to determining that a recovery time satisfies a recovery time threshold.

23. (canceled)

24. (canceled)

25. (canceled)

26. (canceled)
